(19) United States
(12) Patent Application Publication
(10) Pub. No.: US 2013/0123797 A1
(43) Pub. Date: May 16, 2013

(54) SINGLE HAND SEMI-AUTOMATIC TISSUE MORCELLATOR

(71) Applicant: STEVE LIVNEH, AMHERSTBURG (CA)

(72) Inventor: STEVE LIVNEH, AMHERSTBURG (CA)

(21) Appl. No.: 13/662,163

(22) Filed: Oct. 26, 2012

Related U.S. Application Data
(60) Provisional application No. 61/551,554, filed on Oct. 26, 2011.

(51) Int. Cl. A61B 17/22

(52) U.S. Cl. CPC A61B 17/22031
USPC 606/114

(57) ABSTRACT

A morcellator is described which may be operated with one hand, thus providing semi-automatic functionality, for the reduction of solid tissue into smaller pieces, which may then transported and emptied into a specimen bag. The morcellator eliminates the need for stand-alone grasping forceps that are currently used to grasp and pull the tissue against a sharp rotating tubular conduit. The grasping, cutting and transporting of the tissue may be accomplished via the handheld morcellator which provides triggers and control buttons, thus eliminating conventional handheld graspers and foot switches. The surgeon can now hold the scope and visualize the morcellation arena, thus eliminating the need for a second surgeon presently used for this purpose. Moreover, the ability to perform morcellation single handedly, while holding the scope, improves control and avoids wasted time compared to the procedure when it is done by two surgeons.

### CROSS-REFERENCE TO RELATED APPLICATION

The instant application claims priority to U.S. Provisional Patent Application Ser. No. 61/551,554, filed Oct. 26, 2011, pending, the entire specification of which is expressly incorporated herein by reference.

### FIELD OF INVENTION

The present invention relates generally to morcellators, and more specifically to new and improved morcellators for use in minimally invasive surgery.

### BACKGROUND OF INVENTION

General surgery, endoscopy, and especially laparoscopic endoscopy have been rapidly growing practices in the past decades. In many cases of minimally invasive surgery, there is a need to remove large pieces of tissue such as tumors, and sometimes even a whole organ, such as the uterus, kidney, and/or the like.

To facilitate this task, a device generally known as a morcellator has been employed. Accordingly, the morcellator is a device that is typically used for the task of tissue size reduction and transportation. The morcellator is typically a powered (e.g., electrically) device that is used to reduce large sections of tissue into smaller pieces. These smaller pieces are then transported out of the patient's body, typically through the morcellator shaft, resulting in a generally small scar and minimal injury to the patient (as compared to conventional open surgical procedures that would require a relatively large incision to be made in the patient's body in order to access the area of the tissue/organ to be excised or removed).

Current morcellators designs typically require the user, usually a surgeon, to use both hands and one foot to effectively operate the device. For example, one hand generally holds the morcellator with its shaft inside the body cavity, while the other hand generally operates a grasping device, used to operate through the morcellator's shaft and retrieve tissue pieces through the rotating, distally sharp, shaft. The cut pieces are then placed in a collecting container, e.g., for pathological review. The on/off activation of current morcellators is typically done via foot-switch controls. Thus, the surgeon is forced to constantly and repeatedly use both hands and at least one foot during the morcellation procedure. This imposes inconvenience and fatigue on the surgeon and the result is usually slow performance which translates to higher costs for the organ removal/morcellation portion of the surgical procedure.

Accordingly, there exists a need for new and improved morcellators that overcome at least one of the aforementioned disadvantages.

### SUMMARY OF THE INVENTION

In accordance with the general teachings of the present invention, new and improved morcellators are provided for use in minimally invasive surgery.

More specifically, the present invention provides an endoscopic morcellator that can be operated with one hand, thus providing semi-automatic functionality for the reduction of solid tissue (e.g., such as tumors, as well as organs such as the uterus, kidney and/or the like) into relatively small pieces, which are then transported and emptied into a specimen bag, e.g., for later pathological review.

The morcellator of the present invention eliminates the need for stand-alone grasping forceps that are currently used to grasp and pull the tissue against a sharp rotating tubular conduit in conventional morcellators. The morcellator of the present invention provides a system to perform such tasks singlehandedly, with greater convenience and efficiency.

The morcellator of the present invention was developed, in part, as a response to numerous complaints from surgeons regarding fatigue and slowness in connection with current morcellator designs. The new and improved morcellator designs of the present invention provide a major improvement to the morcellation of tissue and organs, e.g., by providing a system to perform the task by using a single hand operated morcellator. These devices of the present invention perform the grasping, cutting and transporting of the tissue via a hand held morcellator that provides triggers and control buttons, thus eliminating the need for a hand held grasper and foot switches.

The morcellator designs of the present invention also allows for the automatic collection of the morcellated tissue into a designated container/specimen bag.

By way of the present invention, the surgeon now can hold the scope and visualize the morcellation arena, relieving the second surgeon presently used for this purpose. Moreover, the ability to perform morcellation single handedly while holding the scope improves control and avoids wasting time, as compared to the conventional procedure when it is done by two surgeons. Accordingly, morcellation time is shortened, fatigue is reduced, and the leading surgeon's control of the process is much improved.

In accordance with a first embodiment of the present invention, a morcellator system for morcellating tissue is provided, comprising:

a conduit member including an end potion having a cutting surface formed thereon, wherein the conduit member includes an area defining a through bore extending therethrough;

a jaw assembly including a pair of pivotable jaw members, wherein at least a portion of the jaw assembly is positioned proximate to the end portion of the conduit member; and

an auger assembly at least partially received in the through bore of the conduit member, wherein at least a portion of the auger assembly is positioned proximate to the end portion of the conduit member.

In accordance with a second embodiment of the present invention, a morcellator system for morcellating tissue is provided, comprising:
a conduit member including an end potion having a cutting surface formed thereon, wherein the conduit member includes an area defining a through bore extending therethrough;
a jaw assembly including a pair of pivotable jaw members, wherein at least a portion of the jaw assembly is positioned on an exterior surface of the conduit member proximate to the end portion of the conduit member;
an auger assembly at least partially received in the through bore of the conduit member, wherein at least a portion of the auger assembly is positioned proximate to the end portion of the conduit member;
a first actuation system selectively operable to cause the jaw assembly to extend distally away from the end portion of the conduit member;
a second actuation system selectively operable to cause the jaw members to pivot to an open position; and
a third actuation system selectively operable to cause the jaw members to pivot to a closed position.

In accordance with a third embodiment of the present invention, a morcellator system for morcellating tissue is provided, comprising:
a conduit member including an end potion having a cutting surface formed thereon, wherein the conduit member includes an area defining a through bore extending therethrough;
a jaw assembly including a pair of pivotable jaw members, wherein at least a portion of the jaw assembly is positioned on an exterior surface of the conduit member proximate to the end portion of the conduit member;
an auger assembly at least partially received in the through bore of the conduit member, wherein at least a portion of the auger assembly is positioned proximate to the end portion of the conduit member;
a handle member operably associated with the conduit member, jaw assembly and the auger assembly;
a first actuation system operably associated with the handle member and selectively operable to cause the jaw assembly to extend distally away from the end portion of the conduit member;
a second actuation system operably associated with the handle member and selectively operable to cause the jaw members to pivot to an open position;
a third actuation system operably associated with the handle member and selectively operable to cause the jaw members to pivot to a closed position;
a fourth actuation system selectively operable to cause the jaw assembly to recede proximally towards the end portion of the conduit member; and
a fifth actuation system operably associated with the handle member and selectively operable to cause the conduit member and the auger assembly to rotate, wherein the conduit member and the auger assembly counter rotate with respect to one another.

Further areas of applicability of the present invention will become apparent from the detailed description provided hereinafter. It should be understood that the detailed description and specific examples, while indicating the preferred embodiment of the invention, are intended for purposed of illustration only and are not intended to limit the scope of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1a illustrates a perspective view of an electromechanical morcellator, wherein the left handle panel has been removed for purposes of clarity of illustration, in accordance with a first embodiment of the present invention;

FIG. 1b illustrates a sectional view of the morcellator depicted in FIG. 1a, in accordance with a second embodiment of the invention;

FIG. 1c illustrates an elevational view of the right side of the morcellator depicted in FIG. 1a, in accordance with a third embodiment of the invention;

FIG. 2 illustrates a perspective view of a rack and motor assembly of the morcellator depicted in FIG. 1a, in accordance with a fourth embodiment of the present invention;

FIG. 3 illustrates an exploded view of a rack and motor assembly of the morcellator depicted in FIG. 1a, in accordance with a fifth embodiment of the present invention;

FIG. 4 illustrates a jaw assembly of the morcellator depicted in FIG. 1a, in accordance with a sixth embodiment of the present invention;

FIG. 5 illustrates a detailed view of a gear system of the morcellator depicted in FIG. 1a, in accordance with a seventh embodiment of the present invention;

FIG. 6 illustrates an exploded view of an auger assembly of the morcellator depicted in FIG. 1a, in accordance with an eighth embodiment of the present invention;

FIG. 7 illustrates a perspective view of a tube member of the morcellator depicted in FIG. 1a, in accordance with a ninth embodiment of the present invention;

FIG. 8 illustrates a perspective view of a tip assembly of the morcellator depicted in FIG. 1a, in accordance with a tenth embodiment of the present invention;

FIG. 9 illustrates a perspective view of an alternative electromechanical morcellator, wherein the left handle panel has been removed for purposes of clarity of illustration, in accordance with an eleventh embodiment of the present invention;

FIG. 10 illustrates a perspective view of a rack system of the alternative morcellator depicted in FIG. 9 in accordance with a twelfth embodiment of the present invention;

FIG. 11 illustrates an elevational view of the alternative electromechanical morcellator depicted in FIG. 9, in accordance with a thirteenth embodiment of the present invention; and

FIGS. 12a-12d depict fragmentary views of a morcellator, in accordance with the general teachings of the present invention, advancing towards a piece of tissue (FIG. 12a), about to grasp a piece of tissue (FIG. 12b), grasping a piece of tissue (FIG. 12c), and then morcellating a piece of tissue (FIG. 12d), in accordance with a fourteenth embodiment of the present invention.

The same reference numerals refer to the same parts throughout the various Figures.

### DETAILED DESCRIPTION OF THE INVENTION

The following description of the preferred embodiment(s) is merely exemplary in nature and is in no way intended to limit the invention, or uses.

The morcellators of the present invention provides greatly improved performance over conventional morcellators by offering the following main advantages:

(1) A pair of integral grasper jaws that may be advanced, opened, and/or closed onto the target tissue and then be caused to recede proximally automatically, thus forcing the tissue against a sharp, rotating, tubular conduit, to then be reduced to relatively smaller cylindrical portions. This integral grasper feature eliminates the need for the stand-alone graspers that are currently used in conventional morcellators. Accordingly, the morcellator of the present invention frees up one of the surgeon's hands;

(2) An internal auger/cutter that aids in grasping the tissue internally as it approaches the tubular conduit, and then transporting the relatively smaller cylindrical portions proximally towards a cutter and into a specimen container. The auger/cutter rotates in counter direction to that of the tubular conduit;

(3) The morcellator of the present invention allows for automatic collection of the morcellated tissue into a specimen container;

(4) All of the primary controls of the grasping jaws and morcellation may be performed with a single hand. Additionally, all of the primary control features may be placed ergonomically on the handle of the morcellator. The need for conventional foot switch controls is thus eliminated; and

(5) The morcellator of the present invention is also provided with a system to rotate/align the jaws at the surgeon's discretion.

By way of a non-limiting example, the surgeon will typically operate the morcellator of the present invention as follows:

The morcellator will typically be inserted with the jaws in a closed position into an insufflated laparoscopic cavity through a typically dedicated cannula, preferably having a non-return valve to maintain sufficient insufflations pressure;

By depressing the jaws advancement trigger, the jaws and corresponding supporting arms and links will slide distally, ending with the jaws' activation arms stopping a short distance (e.g., 2-4 mm) behind the tube's distal end;

The jaws may then be opened at any time, e.g., by depressing the jaws opening trigger;

The jaws' orientation may be aligned by the surgeon by manipulating a rotation knob;

The surgeon then closes the jaws and hence grasps the target tissue to be morcellated;

The surgeon then releases a ratchet or like device (e.g., in the mechanical version of the present invention) and depresses the morcellation trigger simultaneously. A return spring or a morcellation feed motor pulls the jaws assembly with the grasped tissue proximally, thus having the tissue cut by the rotating tube's sharp distal end and further pulled into the tube by an internal counter rotating auger; and

The transported, morcellated tissue then exits through an opening in the handle (e.g., one formed on a left panel thereof) of the morcellator and into an optional attached specimen container.

The following is a more detailed description of the design and operation of the various morcellator designs of the present invention.

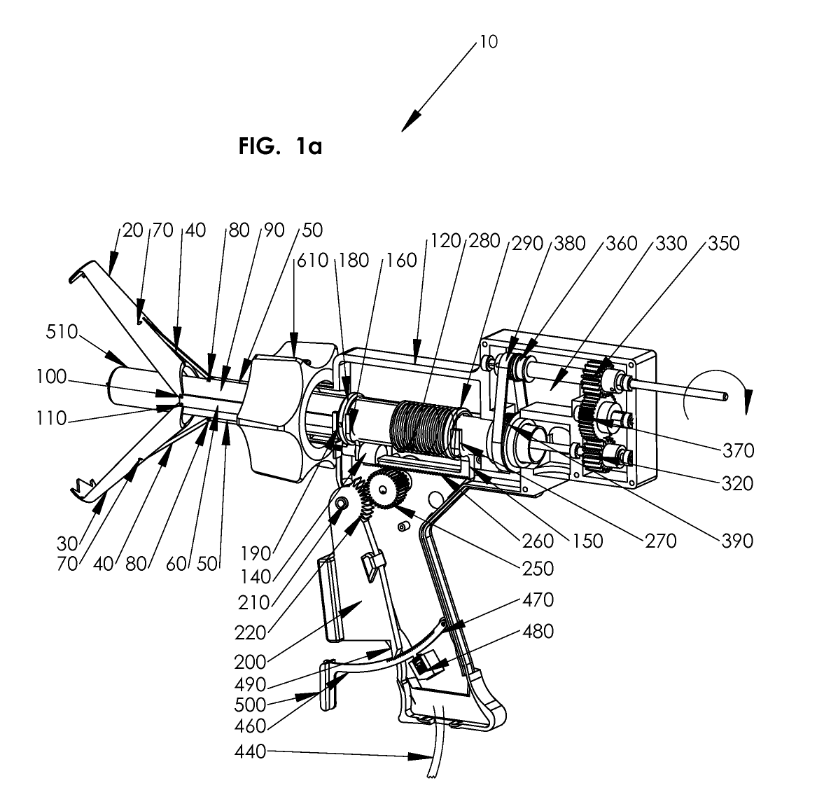

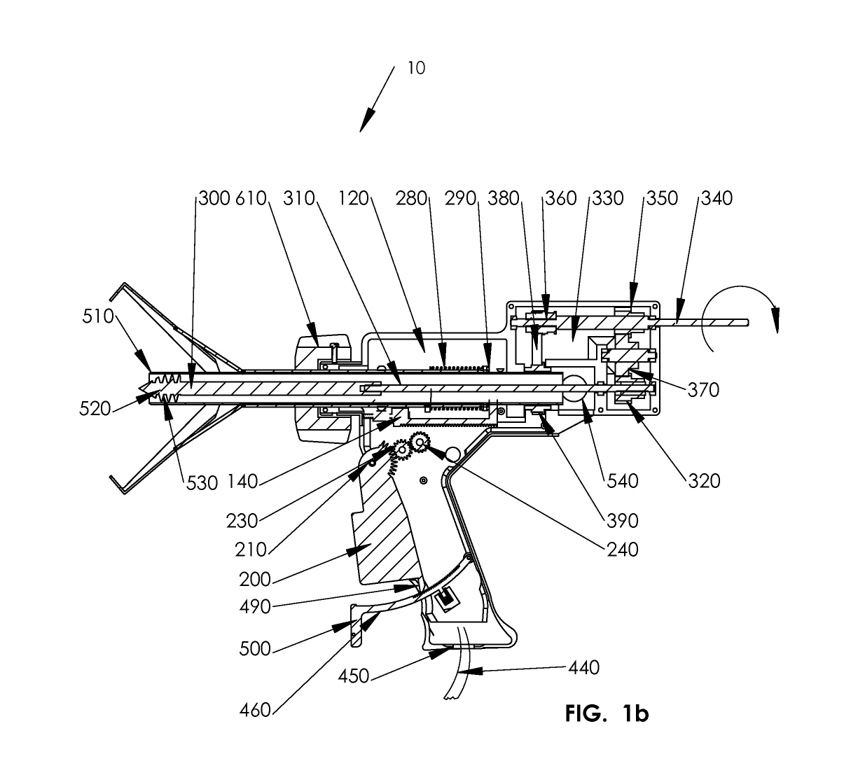

Referring to FIGS. 1a-1c, there is shown a morcellator generally at 10. In this view, a left handle panel member 130 (e.g., see FIG. 1c) has been removed for purposes of illustration and clarity.

The morcellator 10 may be provided with an upper grasping jaw member 20 and a lower grasping jaw member 30. The jaw members 20, 30, respectively, may be used to grasp the tissue/organ to be morcellated as previously described.

By way of a non-limiting example, jaw members 20, 30, respectively, may be supported by support link members 40, which may be attached to jaw members 20, 30, respectively, via pin members 70. The support link members 40 may be connected by control arm members 50 positioned radially around a tube member 90 and connected to the support link members 40 via pin members 80. The jaw members 20, 30, respectively, may be rotatably hinged on activation arm members 60 (it should be noted that in this view only the left jaw member's activation arm is shown in FIG. 1a) via pin members 100, 110, respectively.

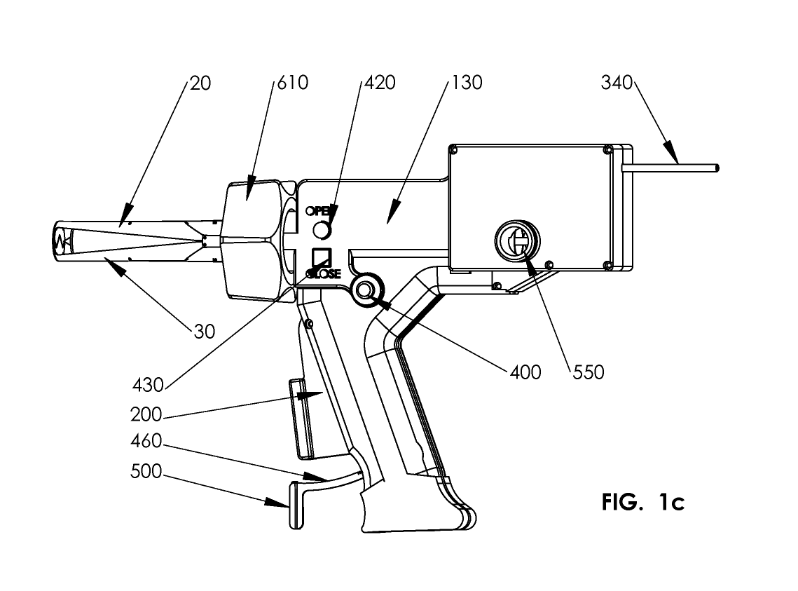

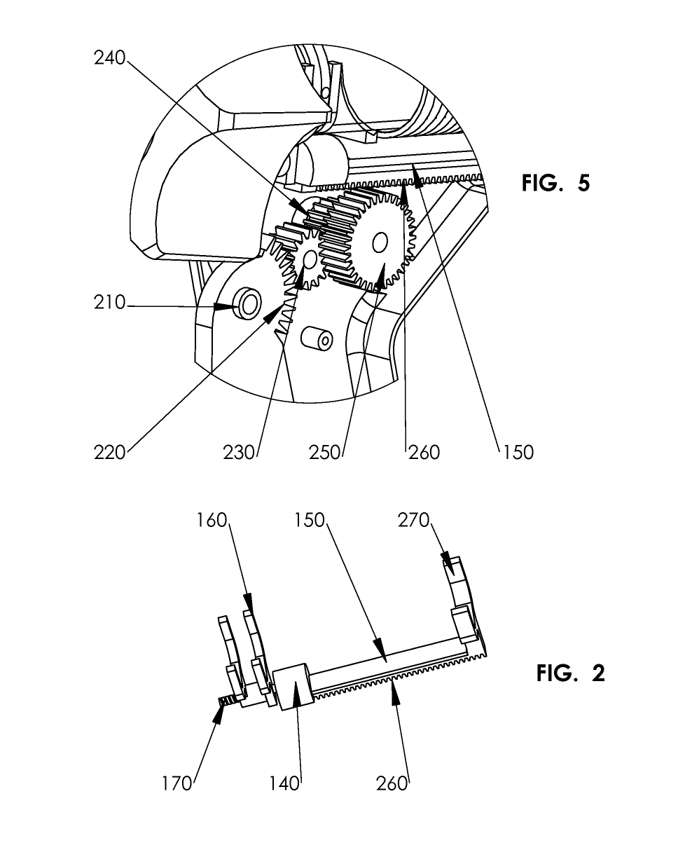

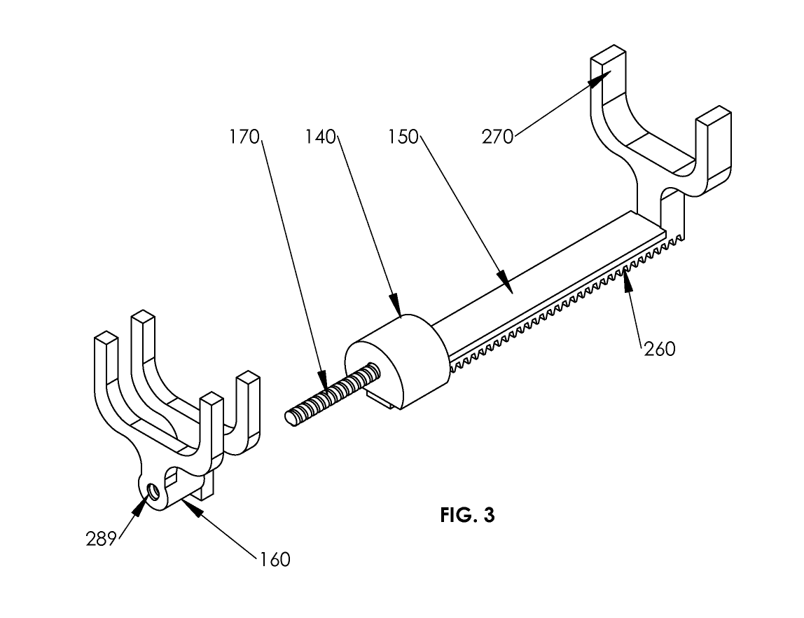

With reference to FIGS. 2 and 3, and by way of a non-limiting example, jaw members 20, 30, respectively, may be opened and closed via an electric motor 140, which may be attached, e.g., permanently, to a rack member 150. The electric motor 140 may push/pull a slingshot member 160 by the rotation of a threaded shaft member 170. The slingshot member 160 may have an area defining a threaded hole 289 that may be engaged with threaded shaft member 170 of electric motor 140.

Referring again to FIGS. 1a-1c, activating electric motor 140 may cause the push/pull of a ring connector member 180, which may be rotatably retained in slingshot member 160. Both the jaw member's activation arms 60 may be connected to ring member 180 by pin members 190. Thus, by activating electric motor 140, slingshot member 160 will either move fore or aft, depending on the motor's rotation, and cause either an advance or retreat of activation arm members 60 and hence cause either the opening or closing of jaw members 20, 30, respectively, simultaneously.

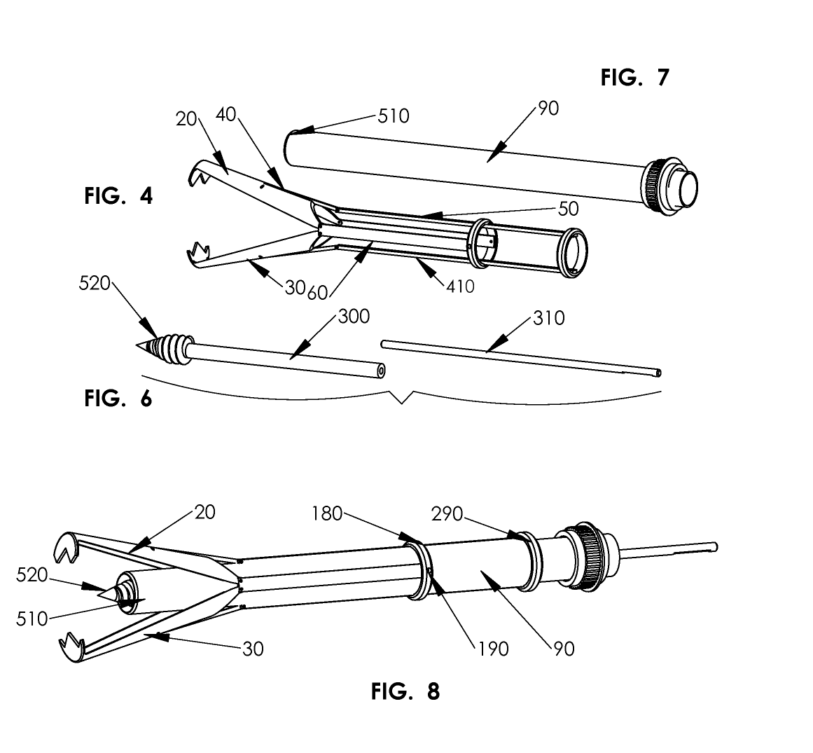

Referring to FIGS. 1a-1c and 4, and by way of a non-limiting example, a jaw assembly 410 may be advanced distally/forward in order to grasp a piece of tissue to be morcellated. For example, advancing jaw assembly 410 distally may be achieved by depressing an advancement trigger 200. Advancement trigger 200 may pivot on a pin member 210 and may include, for example, a permanently attached/integral gear portion 220 that may engage with an idling pinion member 230 (e.g., see FIGS. 1b and 5), which may be engaged with a minor pinion member 240. Minor pinion member 240 may be permanently attached to a major pinion member 250. The resulting rotation of major pinion member 250 may cause a linear movement of rack member 150 distally/forward as major pinion member 250 is engaged with the teeth 260 on rack member 150. Rack member 150 may have a rear slingshot portion 270. Slingshot portion 270 may move distally, pushing an assembly ring member 290 and hence compressing a compression spring member 280 and simultaneously pushing forward jaw assembly 410, i.e., jaw members 20, 30, respectively, control link members 40, control arm members 50, and activation arm members 60. The advanced jaw assembly 410 may be limited in forward movement by design and may stop short of the distal end of tube member 90.

Referring to FIGS. 1b and 6-8, an auger assembly 300 may be rotatably positioned or received within tube member 90 and may be permanently connected via its shaft member 310 to an auger pinion member 320, positioned within a transmission box 330. A power input shaft member 340 (e.g., connected to an electric motor or an electric motor flexible shaft drive (both of which are not shown)) may enter transmission box 330 and may carry an auger drive-pinion member 350, e.g., permanently attached to a shaft member 340 and an upper pulley member 360 to provide for tube member 90 rotation. Power may be transmitted to auger member 300 via an idler pinion member 370 which may be engaged between pinion members 320 and 350. Power may be transmitted to tube member 90 via a toothed belt member 380 and pulley member 390, e.g., permanently attached to tube member 90. Thus, by depressing the morcellation on-off button 400 (e.g., see FIG. 1c), power may be delivered via shaft member 340 into transmission box 330, resulting in simultaneous counter rotation and different rotation directions for both tube member 90 and auger member 300. By way of a non-limiting example, two jaw control buttons may be provided. For example, a top button 420 may be depressed to open the jaw members and a bottom button 430 may be depressed to close the jaw members. Buttons 400, 420 and 430 may be wired to a printed circuit board (not shown), positioned within handle member 120. The printed circuit board may be electrically connected to a cable member 440, e.g., entering handle member 120 via an area defining an opening 450. Cable member 440 may connect to an external power source (e.g. an electrical outlet, power pack, battery, and/or the like) and feed the printed circuit board.

Referring to FIGS. 1a-1c, and by way of a non-limiting example, a ratchet member 460 may be pivotally attached to a pin member 470 and may be normally (except when morcellation takes place) securely locked with a tooth member 490, e.g., permanently attached to advancement trigger member 200. Depressing a ratchet lever member 500 may cause disengagement of ratchet member 460 from tooth member 490 and hence the simultaneous pull-back of the jaw members assembly by compression spring member 280.

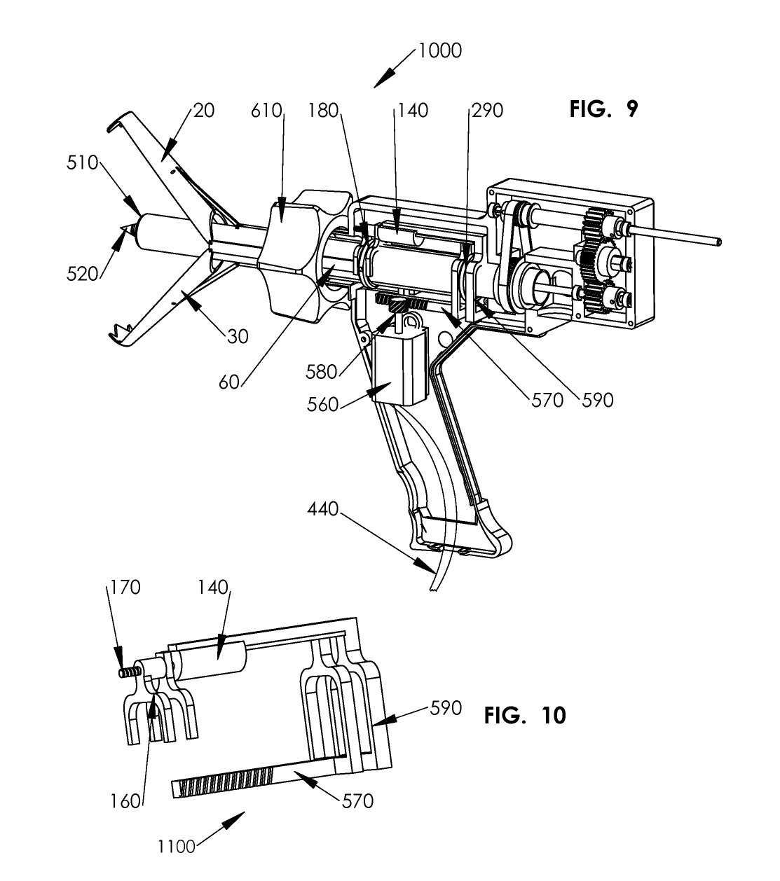

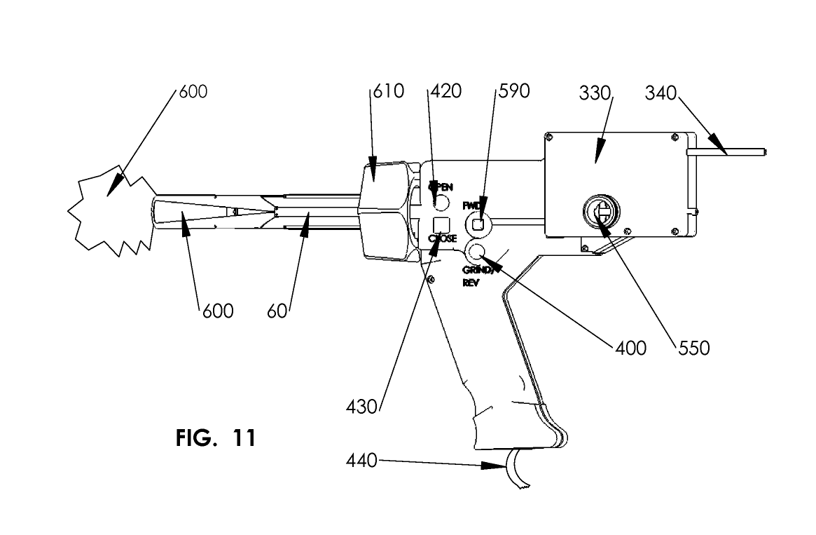

The present invention also provides another variant of the morcellator, generally referred to as an “all electric controls” variation. Referring to FIGS. 9-11, there is shown a perspective view of such an “all electric” morcellator 1000 (in this view, a left handle panel member (e.g., see FIG. 11) has been removed for purposes of illustration and clarity) and the rack system 1100 that is generally required for its operation. FIG. 11 shows an elevational view of the alternative morcellator 1000.

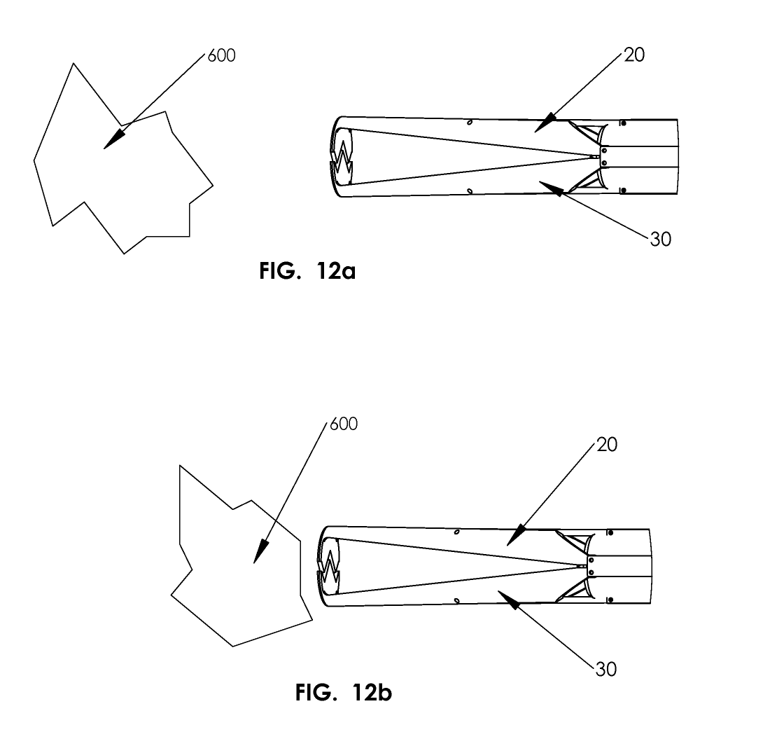

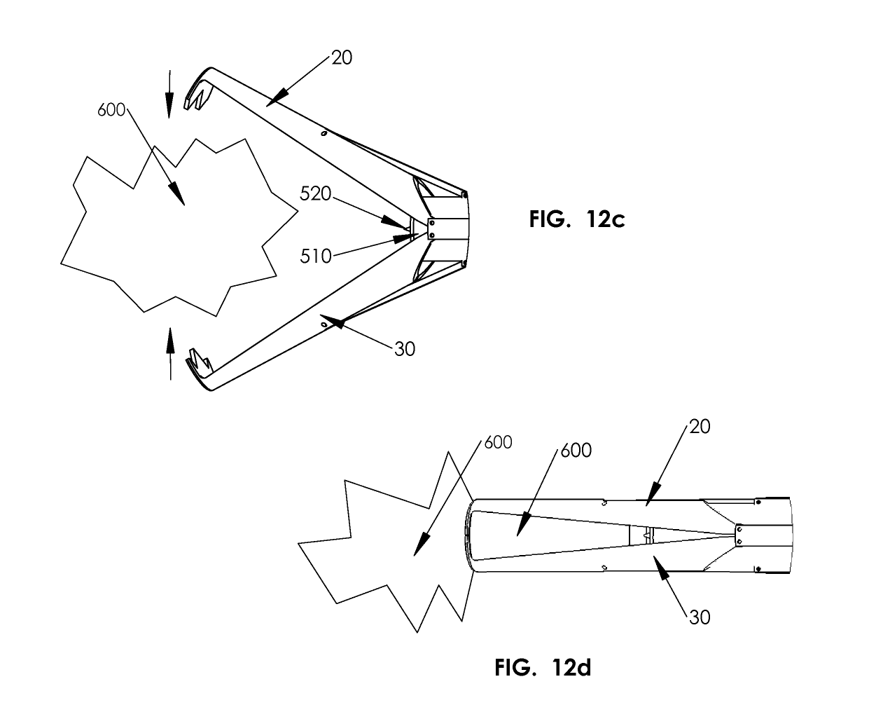

In this variant, there are no mechanical triggers. That is, the jaw assembly 410 moves fore and aft due to the activation of the jaw assembly movement motor 560. The pinion member 580 at the end of the shaft of the motor 560 causes the movement of rack assembly 570 and hence the advancement/retreat of the jaw assembly 410 via a double fork structure 590 that retains ring member 290 that connects the jaw assembly 410. Jaw activation motor 140 may be moved to its upper location as shown and a double slingshot member 160, facing downwardly, may retain ring member 180 that connects to the jaw members 20, 30, respectively, via activation arm members 60. Referring to FIG. 11, there is shown control buttons 420 and 430 for opening and closing the jaws members 20, 30, respectively, and button 400 for the morcellation process. When button 400 is depressed, mechanical power enters transmission box 330 via shaft member 340 and, as a result, tube member 90 and auger member 300 counter-rotate simultaneously. At the same time, powered jaw assembly movement motor 560 is rotating so as to cause rack assembly 570 to move proximally, thus forcing the grasped tissue 600 towards the sharp distal end 510 of tube member 90 (e.g., see FIGS. 12a-12d).

A rotation knob member 610 may be provided for both variants of the morcellators of the present invention. Rotation knob member 610, e.g., once depressed tangentially by the surgeon's finger, may rotate jaw assembly 410 correspondingly and align the jaws as per the surgeon's preference at this time. That is, the orientation of the jaw assembly may be rotated or otherwise manipulated so as to suit a particular need of the surgeon.

The following description will further illustrate a sample morcellation process, in accordance with the general teachings of the present invention, wherein it is assumed that the morcellator of the present invention has already been inserted into the patient's insufflated body cavity. That is, the surgeon has inserted the morcellator shaft into the patient's body cavity through a cannula, for example.

The surgeon may hold the morcellator handle in one of his hands, wherein the jaws are preferably in the closed position. The surgeon may then depress trigger member 200 and thus advance the closed jaw assembly 410 forward towards the area of the target tissue to be morcellated (e.g., see FIG. 12a). Once the shaft appears in the field of view of an endoscope (e.g., see FIG. 12b), the surgeon may then depress the “open” button 420 that activates electric motor 140, thus resulting in the opening of the jaw assembly 410, i.e., the jaw members 20, 30, respectively, rotatingly move apart from one another. The surgeon may then place the jaw members 20, 30, respectively, within grasping reach of the tissue to be morcellated (e.g., see FIG. 12c) and then depresses the “close” button 430, resulting in the jaw assembly closure and grasping of the tissue to be morcellated (e.g., see FIG. 12d). The surgeon may then release ratchet member 460 and depress morcellation button 400. Power may then be fed into transmission box 330 via shaft member 340, resulting in the simultaneous counter rotation of tube member 90 and auger member 300. The receding jaw assembly forces the tissue against the sharp distal end 510 of tube member 90. The tissue is therefore being sheared into relatively smaller cylindrical portions. The screw portion 520 at the tip of the auger member 300 may thread into the tissue and assist in dragging it rearwardly into a fluted portion 530 (e.g., see FIG. 6) of auger member 300 and thus transport the tissue towards a chamber 540 at the rear of morcellator 10. The morcellated tissue may exit via an area defining an opening 550 and into an optional specimen bag/container that may be attached to opening 550.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A morcellator system for morcellating tissue, comprising:
a conduit member including an end portion having a cutting surface formed thereon, wherein the conduit member includes an area defining a through bore extending therethrough;
a jaw assembly including a pair of pivotable jaw members, wherein at least a portion of the jaw assembly is positioned proximate to the end portion of the conduit member; and
an auger assembly at least partially received in the through bore of the conduit member, wherein at least a portion of the auger assembly is positioned proximate to the end portion of the conduit member.

2. The morcellator system according to claim 1, further comprising a first actuation system selectively operable to cause the jaw assembly to extend distally away from the end portion of the conduit member.

3. The morcellator system according to claim 2, further comprising a second actuation system selectively operable to cause the jaw members to pivot to an open position.

4. The morcellator system according to claim 3, further comprising a third actuation system selectively operable to cause the jaw members to pivot to a closed position.

5. The morcellator system according to claim 1, further comprising a fourth actuation system selectively operable to cause the jaw assembly to recede proximally towards the end portion of the conduit member.

6. The morcellator system according to claim 5, further comprising a fifth actuation system selectively operable to cause the conduit member and the auger assembly to rotate, wherein the conduit member and the auger assembly counter rotate with respect to one another.

7. The morcellator system according to claim 6, further comprising a handle member operably associated with the conduit member, jaw assembly and the auger assembly.

8. The morcellator system according to claim 7, wherein the handle member is operably associated with any of the first, second, third or fourth actuation systems.

9. The morcellator system according to claim 1, wherein at least a portion of the jaw assembly is positioned on an exterior surface of the conduit member proximate to the end portion of the conduit member.

10. The morcellator system according to claim 1, further comprising a rotation system for rotating the jaw assembly relative to the conduit member.

11. The morcellator system according to claim 2, wherein the jaw assembly is extended distally away from the end portion of the conduit member so as to be proximate to a portion of tissue to be morcellated.

12. The morcellator system according to claim 3, wherein the jaw members pivot to an open position so as to assume a position operable to grasp a portion of tissue to be morcellated.

13. The morcellator system according to claim 4, wherein the jaw members pivot to a closed position so as to grasp a portion of tissue to be morcellated.

14. The morcellator system according to claim 5, wherein the jaw members recede proximally towards the end portion of the conduit member so as to cause the grasped tissue to abut the cutting surface of the conduit member.

15. The morcellator system according to claim 6, wherein the conduit member and the auger assembly rotate so as draw the grasped tissue into the through bore of the conduit member and towards a spaced and opposed second end of the through bore.

16. A morcellator system for morcellating tissue, comprising:
a conduit member including an end potion having a cutting surface formed thereon, wherein the conduit member includes an area defining a through bore extending therethrough;
a jaw assembly including a pair of pivotable jaw members, wherein at least a portion of the jaw assembly is positioned on an exterior surface of the conduit member proximate to the end portion of the conduit member;
an auger assembly at least partially received in the through bore of the conduit member, wherein at least a portion of the auger assembly is positioned proximate to the end portion of the conduit member;
a first actuation system selectively operable to cause the jaw assembly to extend distally away from the end portion of the conduit member;
a second actuation system selectively operable to cause the jaw members to pivot to an open position; and
a third actuation system selectively operable to cause the jaw members to pivot to a closed position.

17. The morcellator system according to claim 16, further comprising a fourth actuation system selectively operable to cause the jaw assembly to recede proximally towards the end portion of the conduit member.

18. The morcellator system according to claim 17, further comprising a fifth actuation system selectively operable to cause the conduit member and the auger assembly to rotate, wherein the conduit member and the auger assembly counter rotate with respect to one another.

19. The morcellator system according to claim 18, further comprising a handle member operably associated with the conduit member, jaw assembly and the auger assembly.

20. The morcellator system according to claim 19, wherein the handle member is operably associated with any of the first, second, third or fourth actuation systems.

21. The morcellator system according to claim 16, further comprising a rotation system for rotating the jaw assembly relative to the conduit member.

22. The morcellator system according to claim 16, wherein the jaw assembly is extended distally away from the end portion of the conduit member so as to be proximate to a portion of tissue to be morcellated.

23. The morcellator system according to claim 16, wherein the jaw members pivot to an open position so as to assume a portion operable to grasp a portion of tissue to be morcellated.

24. The morcellator system according to claim 16, wherein the jaw members pivot to a closed position so as to grasp a portion of tissue to be morcellated.

25. The morcellator system according to claim 17, wherein the jaw members recede proximally towards the end portion of the conduit member so as to cause the grasped tissue to abut the cutting surface of the conduit member.

26. The morcellator system according to claim 18, wherein the conduit member and the auger assembly rotate so as draw the grasped tissue into the through bore of the conduit member and towards a spaced and opposed second end of the through bore.

27. A morcellator system for morcellating tissue, comprising:
a conduit member including an end potion having a cutting surface formed thereon, wherein the conduit member includes an area defining a through bore extending therethrough;
a jaw assembly including a pair of pivotable jaw members, wherein at least a portion of the jaw assembly is positioned on an exterior surface of the conduit member proximate to the end portion of the conduit member;
an auger assembly at least partially received in the through bore of the conduit member, wherein at least a portion of the auger assembly is positioned proximate to the end portion of the conduit member;
a handle member operably associated with the conduit member, jaw assembly and the auger assembly;
a first actuation system operably associated with the handle member and selectively operable to cause the jaw assembly to extend distally away from the end portion of the conduit member;
a second actuation system operably associated with the handle member and selectively operable to cause the jaw members to pivot to an open position;
a third actuation system operably associated with the handle member and selectively operable to cause the jaw members to pivot to a closed position;
a fourth actuation system selectively operable to cause the jaw assembly to recede proximally towards the end portion of the conduit member; and
a fifth actuation system operably associated with the handle member and selectively operable to cause the conduit member and the auger assembly to rotate, wherein the conduit member and the auger assembly counter rotate with respect to one another.

28. The morcellator system according to claim 27, further comprising a rotation system for rotating the jaw assembly relative to the conduit member.

29. The morcellator system according to claim 27, wherein the jaw assembly is extended distally away from the end portion of the conduit member so as to be proximate to a portion of tissue to be morcellated.

30. The morcellator system according to claim 27, wherein the jaw members pivot to an open position so as to assume a portion operable to grasp a portion of tissue to be morcellated.

31. The morcellator system according to claim 27, wherein the jaw members pivot to a closed position so as to grasp a portion of tissue to be morcellated.

32. The morcellator system according to claim 27, wherein the jaw members recede proximally towards the end portion of the conduit member so as to cause the grasped tissue to abut the cutting surface of the conduit member.

33. The morcellator system according to claim 27, wherein the conduit member and the auger assembly rotate so as draw the grasped tissue into the through bore of the conduit member and towards a spaced and opposed second end of the through bore.
